(19) United States
(12) Patent Application Publication
(10) Pub. No.: US 2024/0293320 A1
(43) Pub. Date: Sep. 5, 2024

(54) Mechanism for Bioavailable Delivery of Electrolytes

(71) Applicant: Sarah Melissa Mulholland, Lexington, KY (US)

(72) Inventors: Sarah Melissa Mulholland, Lexington, KY (US); Juan Jorge Ayala, Healdsburg, CA (US); Chad Michael Goodwin, Seattle, WA (US)

(21) Appl. No.: 18/593,290

(22) Filed: Mar. 1, 2024

Related U.S. Application Data
(60) Provisional application No. 63/449,720, filed on Mar. 3, 2023.

(51) Int. Cl. A61K 9/127; A61K 9/00; A61K 31/00

(52) U.S. Cl. CPC A61K 9/1277; A61K 9/0056; A61K 31/658

(57) ABSTRACT

A method and process for creating an electrolyte based liposome powder. The process involves dissolving electrolyte rich salts in water, adding in various concentrations, emulsifiers such as maltodextrines and gum acacia, adding essential oils, mixing and homogenizing to specified levels, heating and cooling for defined times and to desired temperatures, all to create a liposome with an electrolyte core and an oil based lipid. Then drying through specified processes to create a liposome powder that can be used in water as a soak bath, in water as an electrolyte replacement drink, in oils for a liposome electrolyte cream, and in gelatins for an electrolyte edible gummy.

### CROSS REFERENCE TO RELATED APPLICATIONS

This application claims priority to U.S. provisional application Ser. No. 62/449,720, filed on Mar. 3, 2023, and fully incorporated herein by reference.

### STATEMENT REGARDING FEDERALLY SPONSORED RESEARCH OR DEVELOPMENT

Not Applicable

### The Names of the Parties to a Joint Research Agreement

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates to the process and mechanisms for the production of electrolyte based liposomes for the bioavailable delivery of electrolytes. More particularly the invention relates to the production of liposomes for the transdermal delivery of electrolytes.

### Description of the Related Art

Electrolytes are minerals that dissociate into positive and negative ions upon dissolution in water. Electrolytes are utilized in a variety of necessary processes in your body, such as the regulation of chemical reactions, the maintenance of fluid balance balance across the membrane of human cells, and the generation of action potential for cardiac function. Monitoring a human's electrolyte levels are also utilized to diagnose a wide range of medical conditions and diseases. Electrolytes are vital in regulating body pH levels, nerve and muscle function, hydrating the body and even rebuilding damaged tissue. They also assist in transporting nutrients into the cell, as well as removing unusable waste out of the cell. Currently, one of the most optimal methods for introducing electrolytes into the body is via consumption of an appropriate diet. For example, electrolytes can be obtained from foods like chicken, watermelon, and avocado. One can also obtain electrolytes through liquid drinks like 100% fruit juice, coconut water, or sports drinks. However, appropriate levels of certain electrolytes are not straightforward to achieve through the primary diet approach, especially when in regards to high-performance athletes or segments of the population with specific electrolyte deficiencies, originating from dietary constraints. For certain individuals and animals, pharmaceuticals can interfere with the absorption of electrolytes in situations where the regulation of electrolyte balance is already challenging. Hypokalemia is a common electrolyte disturbance, especially in hospitalized patients, indicated by low levels of potassium in the bloodstream. This condition can have various causes, including endocrine disruptions. Infants and Children are often at risk of low electrolyte levels, especially when losing fluids due to short or long-term illness. Other common electrolyte imbalances are exhibited due to dehydration, over hydration, diet, excessive sweating, severe burns, and kidney disease.

In the instance of athletes and sports medicine, Magnesium is a particularly important electrolyte to physical performance and to reducing stress on the body after training. Magnesium is the eleventh most abundant element by mass in the human body and is essential to all cells and around 300 enzymes. Magnesium ions interact with polyphosphate compounds such as ATP (adenosine triphosphate), DNA (deoxyribonucleic acid), and RNA (ribonucleic acid). Hundreds of enzymes require magnesium ions to function. Magnesium compounds are used to stabilize abnormal nerve excitation or blood vessel spasms. Athletes, whether human or animal, require proper magnesium levels before and after a high intensity performance. Research on magnesium status in athletes indicates that even a marginal deficiency can impact exercise performance and can amplify the oxidative stress that occurs during exercise. A deficiency can also impact outcomes in the event of a skeletal, muscle or brain injury. A suboptimal blood magnesium level results in inefficient energy metabolism and decreased endurance in strength training athletes. Athletes who participate in weight class sports or competitive sports both of which might have periodic food restriction (e.g., boxing, wrestling, gymnastics, ice skating), can be particularly vulnerable to inadequate magnesium intake without a properly formulated diet. Studies on athletes show suboptimal magnesium levels can occur well above the 50-percent prevalence seen in the average adult population. “Analysis of published metabolic data indicates that the minimal daily requirement is not 220 to 300 mg. per day, as has been reiterated, or even 5 mg. per kg. per day as has also been suggested, but probably at least 6 mg. per kg. per day” Muscles, bone, and other tissues store about 99 percent of the body's magnesium, with one percent in the serum and red blood cells (RBCs). When serum magnesium decreases, such as during excessive sweating or increased energy production demands, magnesium is pulled from tissue storage to maintain the tightly regulated blood magnesium level. Thus, many low-magnesium side effects might be avoided, such as twitches, muscle weakness, cramps, fatigue, numbness, abnormal heart rhythms, or even seizures by adding magnesium to the body. Replacing magnesium through consumption of high-quality foods and supplements helps restore and maintain serum, red blood cell, and muscle/bone levels.

Another important electrolyte is sulfate which is required by all human cells to function normally. Sulfate is among the most important macronutrients in cells and is the fourth most abundant anion in human plasma (300 μM). Sulfate is the major sulfur source in many organisms, and, due to its hydrophilic nature, this anion cannot passively cross the lipid bilayer of cell membranes, thus all cells require a mechanism for sulfate influx and efflux to ensure an optimal supply of sulfate in the body. The class of proteins involved in moving sulfate into or out of cells is called sulfate transporters. If one assumes that adults whose dietary protein needs are being met will consume a daily intake of 2 g of methionine and 2 g of cysteine, an equal amount of methionine and cysteine would be oxidized, producing 960 mg of sulfur, or 2.8 g/day of inorganic sulfate. A daily intake of inorganic sulfate as high as 1.3 g/day can be obtained from water and other beverages (0.5 g/L×2.6 L/day). A quantity of sulfate greater than this amount would likely be produced daily from the metabolism of methionine and cysteine in food, in addition to that from body protein turnover. An analysis of the sulfate content of various diets using foods purchased at supermarkets suggests a large variation in daily inorganic sulfate intake, ranging from 0.2 to 1.5 g (2.1-15.8 mmol)/day.

One way to intake the proper amount of both magnesium cations and sulfate anions would be to use Epsom Salt (consisting of magnesium sulfate) as a dietary supplement. The current use cases for magnesium sulfate are not however efficient. Magnesium Sulfate (recommendation is two or more cups per bath) is used in bath water, that is one, not hot enough to actually dissolve the particles and two can not penetrate the skin in it's current form. The salts consist of non-ideal particles which are −4-significantly too large in size to enter the epidermis (around 10.5 microns). For the Epsom Salt to actually be effective it would need to obtain particles of smaller size, with high-temperature heating or dissolving/mixing in water for long periods of time is required. Hot water causes the skin to eliminate rather than absorb, which also makes his therapeutic ritual even more difficult to be effective. Although there are many theories of magnesium sulfate (MgSO₄) being an source of electrolytes in water, it is not ideal for epidermis adsorption. To utilize MgSo₄ into the body most effectively in baths, soaks, creams, cosmetics, toiletries, or medicines, the MgSo₄ should be compatible with the other ingredients and the body. Currently, the magnesium and salts can not enter the body because of the human body's excellent design of keeping out toxins. “The barrier function of stratum corneum, which is hardened-flatted-stacked dead cells with 10-20 μm in thickness, provides a waterproof protection (FIG. 4.4.3) and is the most serious problem of inhibition of permeating hydrophilic drug through the stratum corneum by its barrier effect, because the stratum corneum is composed of proteins (79%-90%) and lipids (5%-15%) (Patel and Baria, 2011). The stratum corneum inhibits also to permeate the relatively larger drug molecules into the epidermis for more than 500 Da in molecule size (Bos and Meinardi, 2000). The stratum corneum does have not only a barrier function but also roles of regulating for natural water loss by water evaporation from our skin, pH, and temperature as being 5-30 g/m2 h, pH 5.0-5.4, and 31-33° C., respectively (Yosipovith et al. 1998).” https://www.sciencedirect.com/topics/medicine-and-dentistry/transdermal-drug-administration.

Once MgSO₄ combines with water it becomes Mg2+So42-ions. Magnesium does not react well with cold/warm water and becomes coated in magnesium hydroxide which has a very low insoluble rate. When MgO is combined with hot water it becomes magnesium hydroxide with hydrogen gas sticking to the surface. This will cause the magnesium sulfate to float to the top of a bath. Many Epsom Salt baths on the market today also start with a large particle size that is coated in an essential oil, thus making it even more difficult to enter the skin at its current size. The most effective ways currently to increase solubility of MgSo₄ are to reduce particle size, changing the Ph of the water, higher temperatures allowing the solvents molecules' kinetic energy to rise-thus enabling more collisions with the solute particles and promoting dissolution-none of which happen in a traditional bath soak.

Currently commercial options for magnesium absorbation in the bath consist of Magnesium Chloride baths. These products are not oil-based but might feel that way to consumers. These bath flakes are not ideal for electrolyte absorption for several reasons. Magnesium chloride should not be used on sensitive areas or mucus membranes, as it can cause redness or irritation, and could also cause respiratory depression, dizziness, and sweating. The Magnesium Chloride gels will not fully absorb. For certain individuals, it is recommended to wash off any magnesium chloride after use, to avoid stinging or a lingering burning sensation on the skin. “Magnesium oil is touted extensively online as a potential curative for many conditions, such as migraines and insomnia. However, the research on topical magnesium is very limited, and there are varying opinions as to the body's ability to absorb it fully through the skin. Magnesium oil has been shown in one small study to alleviate fibromyalgia symptoms, such as pain.” (Kubala, Jillian. “Magnesium Oil Benefits: Forms, Benefits, Uses, and Risks.” Healthline, https://www.healthline.com/health/magnesium-oil-benefits #takeaway. Accessed 26 Jan. 2023.)

### SUMMARY OF THE INVENTION

The invention relates to a method to create a vesicular encapsulation of oil- and water-soluble therapeutics, to create liposomes that are micron and sub micron in size. The large amount of small lipid vesicles that are created gives this method its high transdermal capabilities, and in a small dose of around 10-20 grams per bath. The specific encapsulation consists of a oil-in-water micelle in which the oil phase contains much smaller water-in-oil “reverse” micelles. Gum Acacia is used as an oil-in-water emulsifier, and is a key ingredient for achieving the encapsulation. For proper results, the quantity of “Oil-Phase” product and Gum Acacia needs to be balanced for long-term particle stability. The internal electrolyte water phase (called salty water in our experiment) is similarly connected to the oil-soluble emulsifier, Glycerol Monostearate (GMS). Maltodextrin may be used to increase the particle size of the spray-dried product.

This invention relates to liposome creation for electrolyte and nutrient delivery, and specifically to methods for preparation of these Liposomes and their use. The invention relates to a method for preparing stable liquid emulsion forms of electrolytes. In the most basic embodiment this involves providing a first volume of distilled water, then dissolving maltodextrin and gum acacia in the distilled water, mixing for fifteen minutes, placing the mixture in a sealable container and storing for 24 hours at a temperature below 50 Degrees Celsius to create a “water phase” solution; next providing a second volume of distilled water then dissolving Epsom salt in second volume of distilled water and mixing for between 2 and 6 hours to create a “salty water phase” solution, then storing this at room temperature of subsequent use; next providing a warmed vessel warmed to 60 Degrees Celsius, add a volume of coconut MCT oil, then mix the desired essential oils to the warmed vessel, them mix in a volume of cannabinoids, and then begin mixing this solution, add glycerol monostearate to the solution being mixed, and continue to mix for 15 minutes to create a “oil phase” solution, keeping the solution below 80 Degrees Celsius, coving the mixture while preparing other steps; now mixing the “oil phase” solution, adding the “salty water phase” solution to the mixing “oil phase” solution, and continue mixing for at least fifteen minutes to create a “salty water in oil” emulsion, cover and store this emulsion at 60 Degrees Celsius; homogenize the “salty water in oil emulsion in a high pressure homogenizer at between 10,000 to 20,000 psi, then add a volume of heated coconut MCT oil at 75 Degrees Celsius, then pass this mixture through the high pressure homogenizer three times; placing the “water phase” solution in a vessel, placing the vessel into a cool bath, begin shear mixing the cooled water phase, adding the “salty water in oil” emulsion at a rate of 1 g/see, continue mixing for at least 15 minutes, remove any substance that floats to the top, to create a “salty water in oil in water” phase; homogenize the “salty water in oil in water” emulsion in a high pressure homogenizer at between 10,000 to 20,000 psi, and passing the emulsion through the homogenizer three times, coving and storing the emulsion at 22 Degrees Celsius, drying the emulsion to create a water soluble powdered liposome. This liposome is electrolyte rich and can now be used to transdermally introduce electrolytes into the body. It can be added to water to create a soak, added to oils and gels to create a cream, or added to gelatin to create an edible gummy.

### BRIEF DESCRIPTION OF THE DRAWINGS

FIG. 1 is a photograph of a microscopic image showing the liposomes of the present invention.

FIG. 2 is a photograph of a microscopic image of the liposomes of the present invention with a scale to show the size of the liposomes.

FIG. 3 is is a second photograph of a microscopic image of the liposomes of the present invention with a scale to show the size of the liposomes.

### DETAILED DESCRIPTION OF THE INVENTION

Detailed embodiments of the present invention are disclosed herein. It is to be understood that the disclosed embodiments are merely exemplary of the invention, and that there may be a variety of other alternate embodiments. The figures are not necessarily to scale, and some features may be exaggerated or minimized to show details of particular components. Therefore, specified structural and functional details disclosed herein are not to be interpreted as limiting, but merely as a basis for teaching one skilled in the art to employ the varying embodiments of the present invention.

To relieve the bodies' reaction to oral intake for Magnesium Sulfate (laxative, nausea, headache effects) as a supplement and increase absorption, a delivery method that bypasses the digestive system is the best possible solution. Magnesium Sulfate can be absorbed through the skin transdermally, but the rate that this process is typically achieved is low, due to the large size constraints of magnesium sulfate. Furthermore, for optimal transdermal introduction into the body, the magnesium sulfate should be fat-soluble. This method would alleviate reactions for people deficient in such electrolytes due to illness or medications. To achieve this double emulsion delivery method, a micro spherical encapsulation will contain an optimized water-soluble electrolyte composed of any combination of electrolytes (dead sea salt, Epsom salt, magnesium chloride, potassium, etc) along with an oil soluble outer layer containing (vitamins, essential oils, oil based medications, etc). That way electrolytes can be replenished after being lost through sweating or through water loss of the body. Some examples of the vitamins and minerals lost through sweat are vitamin C and potassium. Both examples are important for the body to perform and maintain optimal health. Any combination of water-soluble electrolytes and nutrients can be combined with oil soluble nutrients to create a delivery system that absorbed immediately when taken orally, or transdermal though the dermis when dispersed in water-(warm, cold, or hot) soaked in, swallowed, in cream/serum form and even inhaled. The dual delivery of hydrophilic and hydrophobic compounds make this delivery method exclusively beneficial.

This encapsulation is a liposome, which is defined as being a very tiny, fat-like particle that is made in the laboratory. In medicine, liposomes containing drugs or other substances are used in the treatment of cancer and other diseases. Drugs given in liposomes may have fewer side effects and work better than the same drugs given alone. The liposome size can vary from very small (0.025 μm) to large (2.5 μm) vesicles. Moreover, liposomes may have one or bilayer membranes. Liposomes are promising drug carriers to enable noninvasive transdermal delivery. Transdermal delivery method advantages over hypodermic injections, which are painful, generate dangerous medical waste and pose the risk of disease transmission by needle reuse, especially in developing countries. In addition, transdermal systems can be self-administered. They can provide release for long periods of time (up to one week). These lipids/proliopsomes will then maintain a dispersed suspension for several days/years when added to water. “Charge differences between particle surfaces (e.g., negative to negative) confer stability through charge repulsion, meaning that nanoparticles and colloids tend to remain dispersed in water, forming a suspension.” (“The Environmental Significance of Natural Nanoparticles.” Learn Science at Scitable, 10 Aug. 2022, http://wwww.nature.com/scitable/knowledge/library/the-enviroment-significance-of-nature-nanoparticles-105737311/. Accessed 26 Jan. 2023.) These also improve patient compliance and the systems are generally inexpensive. The resulting transdermal delivery package of essential oils, key vitamins, natural remedies and powerful magnesium salts, other salt forms, etc, which synergistically work to reduce inflammation, manage pain, and promote healing. This means the therapy product does not settle or float allowing for effective treatment during use-evenly dispersed. The magnesium/salt based liposome can be used on open wounds, as well as closed wounds. Antibacterial and anti-inflammatory essential oils, salts, drugs, peptides, many different forms of magnesium including magnesium chloride and Epsom salt, etc are processed inside the liposomes in the nano size range <1 um and below allowing them to stay dispersed in water and interact with the epidermis allowing it to penetrate the skin.

By using the vesicle encapsulation system of the liposome, which alters the pathways in which the human body can accept electrolytes, delivery is improved both through transdermal absorption and oral uptake. In the use of transdermal uptake, the lipophilic outer shell and size (500 nm to 10 um) of the liposome is readily able to interact with the outer layer of the epidermis into the dermis where the absorbed liposomes can be processed into the bloodstream. This allows for general treatment of the body or very specific targeted areas. In terms of recovery, this means there are many more options and opportunities on how to treat an injury or drop in overall electrolytes and nutrients in the body. As the skin is the largest organ on the human body, the body lends itself well to this kind of delivery system where medications interact with certain ions and vitamins. For athletes, this means that, both during pre- or postgame periods, they can get the same electrolyte delivery in warm and/or cold water, including ice baths.

In oral usage, liposomes are biocompatible carriers employed to improve oral bioavailability of drugs and, in addition to the general advantages of nanocarriers for oral delivery, they offer benefits derived from their lipid bilayer structure. They have been successful in improving oral bioavailability of a variety of compounds including peptide and proteins, hydrophilic and lipophilic drugs. These include using appropriate lipid compositions, polymer coating, addition of stabilizing lipids to liposomal structures, preparation of double liposomes and proliposomes, in addition to other innovative methods. Oral administration of medication is the first option when patient compliance is considered. However, many barriers face oral absorption of drugs which limit bioavailability in about 90% of therapeutic agents. While electrolytes are absorbed through passive diffusion and active transport in the stomach and small intestines, respectively, liposome/proliposome delivery would help lead to reducing interactaction with other molecule/medicines currently in the intestinal tract and stomach. Also a benefit includes protectection from pharmaceutics degrading as well as acting as protection from a low pH of the stomach.

A wide variety of ingredients can be used, and are used, in the present invention. The following table, Table 1, sets out the ingredient, its source, its purpose in the present invention, and other facts pertinent to the present invention.

TABLE 1
Sample Ingredient list

| | | Purpose in | |
|---|---|---|---|
| Ingredient | Source | product | Other Facts |
| Gum Acacia | Tree sap from | Oil-in-Water | |
| | Acacia tree | emulsifier | |
| Maltodextrin | Corn | Prevents the | |
| | | spray-dried | |
| | | product from | |
| | | being too fine | |
| Coconut MTC | Coconuts | Solvent/filler | MTC is highly sought |
| (Medium Chain | | | after for consuming as |
| Triglyceride) | | | it is readily turned into |
| Oil | | | energy |
| CBD | Hemp | Pain and | 99.99% pure called |
| (Cannabidiol) | | inflammation | CBD Isolate |
| | | relief, etc | |
| CBG | Hemp | Also pain and | May help skin, brain, |
| (Cannabigerol) | | inflammation, | digestion, and more |
| | | much more in | 99.99% pure called |
| | | different ways | CBG Isolate |
| Eucalyptus | Eucalyptus | Stress relief, | Research has suggested |
| essential oil | globulus tree | scent, | hundreds more benefits |
| | leaves | decongestion. | |
| | | Also anti- | |
| | | inflammatory | |
| Black pepper | Black pepper | Increase Impact | anti-inflammatory, |
| essential oil | seeds | of CBD and CBG | antispasmodic, |
| | (peppercorns) | Cause | diaphoretic, |
| | | warming/tingling | carminative, aperient, |
| | | feeling | antirheumatic, |
| | | | antiarthritic, |
| | | | antibacterial, |
| | | | antioxidant - shown to |
| | | | have potential |
| | | | anticancer |
| GMS (Glycerol | Chemical | Water-in-Oil | Glycerol content used |
| Monostearate) | reactions | emulsifier | before exercise for |
| | between multiple | | cellular hydration, |
| | animal/plant- | | athletic performance |
| | based | | |
| | ingredients. | | |
| Epsom salt | Nature. Lakes. | Supply | Magnesium essential to |
| | | magnesium | recovery and |
| | | | preventing cramps |

The invention is defined initially through the steps of the most preferred embodiment, but it is to be realized that the process is scalable and can be produced on a much larger scale using appropriate industrial equipment. In the most preferred embodiment, the first step in preparing the liposome vesicular encapsulation is the preparation of the “water phase.” This is achieved by first providing the appropriate volume of distilled water in a beaker. In the most preferred embodiment, this is conducted at room temperature of approximately 21 Degrees Celsius, but can be done at standard temperatures of between 15-25 Degrees Celsius. Table 2 shows the components of the water phase as set out in the most preferred embodiment. The second step is providing the appropriate mass of Maltodextrin and Gum Acacia. Next, slowly add first the Maltodextrin until dissolved and then then Gum Acacia to the distilled water while using a homogenizer to mix the components into the water. Next mix with a shear mixer for 15 minutes. For optimal results, ensure that this solution remains below 50 Degrees Celsius. Next, transfer the solution to a sealable container, and allow it to remain at room temperature for at least 24 hours. This allows the maltodextrin to fully hydrate and become useful as an emulsifier. Do not refrigerate, as chilling can cause separation of the ingredients. The solution should be used within 7 days to avoid spoiling and/or mold growth.

TABLE 2
Water Phase (external): 895 g

| | Mass | % of Phase | % of Total |
|---|---|---|---|
| Ingredient | (g) | (by mass) | (by mass) |
| Distilled Water | 581.75 | 65 | 58.20 |
| Gum Acacia | 179.00 | 20 | 17.90 |
| (emulsifier) | | | |
| Maltodextrin | 134.25 | 15 | 13.40 |
| (agglomerator) | | | |

The next step is the “Salty Water Phase”, also known as the electrolyte phase. Introduce the desired amount of distilled water into a beaker. Next select the desired amount of Epsom salt, as set out in Table 3, and add to the distilled water. Add a magnetic stir bar to the beaker and cover, then stir the solution until the solids are fully dissolved in the solvent. Then, continue stirring for several hours more (between 2 and 6 hours) for full dissociation of the solids to occur. Leave the solution covered at room temperature (21 Degrees Celsius) for use in the following steps.

TABLE 3
Salty Water Phase (internal): 5 g

| | Mass | % of Phase | % of Total |
|---|---|---|---|
| Ingredient | (g) | (by mass) | (by mass) |
| Distilled Water | 3.2 | 64 | .32 |
| Epsom Salt | 1.8 | 36 | .18 |

The next step is the preparation of the “Oil Phase.” First, prepare a large beaker in a hot bath set to a controlled temperature of 60 Degrees Celsius. Select the desired amount of coconut MCT oil. Table 4, below, sets out the amount of coconut MCT oil for the most preferred embodiment. Add the coconut MCT oil to the warmed beaker. Next, select the specifically desired essential oils, prepare the desired amounts, and then add these essential oils to the coconut MCT oil in the beaker. Additionally, select the desired mass of the cannabinoids, and add to the solution in the beaker. Then, begin shear mixing the oils together on low-medium speed. While this is being mixed, measure out the desired amount of glycerol monostearate and add to the beaker currently being mixed. Once this emulsifying agent, glycerol monostearate, is added, the mixture will thicken significantly. Shear mix this “oil phase” mixture on high for 15 minutes, to allow the glycerol monostearate to fully dissolve. It is important that this “oil phase” does not heat up to above 80 Degrees Celsius. Cover the “oil phase” to prevent evaporation of essential oils, and allow the “oil phase” to remain for at least 12 hours.

TABLE 4
Oil Phase: 100 g

| | Mass | % of Phase | % of Total |
|---|---|---|---|
| Ingredient | (g) | (by mass) | (by mass) |
| Coconut MCT oil | 55 | 55 | 5.5 |
| (solvent) | | | |
| CBD- Cannabidiol | 20 | 20 | 2 |
| CBG- Cannabigerol | 10 | 10 | 1 |
| Eucalyptus Globulus | 5 | 5 | 0.5 |
| Leaf Oil | | | |
| Piper Nigrum Essential | 5 | 5 | 0.5 |
| Oil | | | |
| Glycerol Monostearate | 5 | 5 | 0.5 |

The next step is to create the Salty Water-in-Oil emulsion. First, ensure that the “oil phase” is at least 60° C. to avoid solidification of any component. Next begin shear mixing the “oil phase” mixture on high using a shear mixer. Next, slowly add the “salty water phase” to the center of the vortex created by the shear mixer at a rate of approximately ˜1 g/sec. At this point, small micelles of the mixture should form rapidly at this point causing the mixture to become opaque. Shear mix the mixture on medium for at least 15 minutes. It is important to ensure that the combined mixture does not exceed 80° C. The shear mixing can be completed in phases, if necessary to keep the temperature from rising. Once mixing is complete, cover the salty water-in-oil emulsion and keep at 60 Degrees Celsius to avoid solidifying. The mixture should optimally be stored between 60 Degrees Celsius and 80 Degrees Celsius. The mixture should appear consistent throughout, with no undissolved solids or additional water out of the overall solution.

The next step is to homogenize the “Salty Water-in-Oil” emulsion, which will decrease the size of micelles such that they can be encapsulated into the next larger micelle to be formed, as well as to increase the stability of the emulsion. For optimal results, in the most preferred embodiment, a high pressure homogenizer is used and the mixture kept at between 10,000-20,000 psi. Next, pass a small amount of heated Coconut MCT oil at 75° C. through the homogenizer to heat the system. Pass the “salty water-in-oil” emulsion through the homogenizer at least three times, though it is to be understood that additional homogenization will not damage the oil. Cover this emulsion once homogenized and return to the hot bath to be maintained at 60° C.

The next step is to create the double emulsion of “Salty water-in-Oil-in-Water.” First, pour the “water phase” (above) into a large beaker. Next, prepare a cool bath for the emulsion to take place in. Begin shear mixing the water phase with a shear mixer on medium-high. Slowly add the “salty water-in-oil” emulsion to the center of the vortex at a rate of approximately 1 g/sec. At this point, micelles should form rapidly, causing the mixture to turn an opaque cream color. Continue shear mix on medium-high for at least 15 minutes. Ensure that the emulsion does not exceed 60 Degrees Celsius. The shear mixing can be completed in phases to ensure that the temperature does not rise above this threshold. . . . This stage is known as the double emulsion state. There should be only one phase visible, and any creamy/oily substance that floats to the top of the mixture is solution from the oil phase that has failed to be encapsulated. This should be removed and discarded using a pipette.

In the next step, the double emulsion will be homogenized to decrease the vesicle size, which will increase emulsion stability and product effectiveness, since transdermal capabilities are dependent on vesicle/particle size. This step will be conducted using a high pressure homogenizer at 10,000-20,000 psi. The double emulsion should be passed through the homogenizer at least three times. Additional homogenization is only beneficial to the final product and will not damage the mixture. Cover the resulting emulsion once homogenized and keep at 22 Degrees Celsius or slightly cooler. Note, that if drying immediately there is no need to cool. Note also that if the emulsion will not be dried right away, cooling will increase shelf life. But if this is the case, do not cool below 10 Degrees Celsius.

The final step is to dry the double emulsion to create the final product. Drying can be done by spray drying, steam drying, on a sheet in an oven, flake drying, or other similar drying methods. At this point the resulting product will a water-soluble powder, a proliposome. It can be further ground if desired to a create a desired particle size. When added to water (cold, warm, or hot) the product will disperse into a suspension that appears cloudy within 60-90 seconds, and will contain the embedded liposome therapeutics.

In one embodiment a hydrogen water core is created in the liposome. This allows magnesium (an electrolyte known for creating digestive issues if taken in too large of a dose) and malic acid to get directly into the blood stream, thus bypassing the digestive system. Hydrogen water has been thoroughly studied, and shown to aid in increasing energy levels, discourages cellular aging, improves muscle recovery after a workout, improves skin health and boosts energy. Malic acid also helps increase energy levels by participating in the citric acid cycle (also known as the Krebs cycle), a process that generates energy in cells by metabolizing carbohydrates, proteins and fatty acids to create adenosine triphosphate. The hydrogen water core in this case would be best combined or enclosed within the lipid, with other drugs or therapeutics to aid in supporting the immune system. This would also increase the acidity in the water, which would aid in the delivery of any therapeutics into the body. The magnesium with malic acid, also known as magnesium malate, in filtered water will be the core of one embodiment of the liposome transdermal delivery system. This would be achieved by replacing hydrogen water with distilled water in the “salty water phase” or the embodiment described above. The best way to create this is to place to 2 grams of malic acid and 4 magnesium rods in a vessel and add boiling water (not distilled water), which will create the hydrogen water. Enclose the vessel and then start reducing the temperature for maximum solubility. This can be done with an ice bath or freezer, though the water should not be frozen, and set for between 1 and 1.5 hours. This mixture will then replace the water in the “salty water phase” described above.

In another embodiment the creation of liposome for electrolyte and nutrient delivery is achieved by the following steps. In the first step, the electrolytes are dissolved in distilled water. To best dissolve water based soluble particles before the polar phase, which can be done using the smallest size particle, so that it has a larger surface area for dissolving or by a heating method. More electrolytes that can be fully dissolved can be encapsulated in the liposome. Next add amino acids, cholesterol, phospholipids for example phosphatidylcholine to one of the for example components in the liposome bilayer. These are thoroughly mixed, in one embodiment through the use of mixed-shear mixing, and create what is called the “therapeutic polar phase” of the process. A wide variety of other polar-soluble therapeutics can be added in this phase as well, including Vitamin C, Vitamin B-12, honey, Aloe vera, collagen, witch hazel, in addition to any other hydrophillic drugs, such as standard pain medications, so long as they can be dissolved in water.

In one embodiment use magnesium (an electrolyte) and malic acid. This will more perfectly dose the concentration of the hydrogen in the core of our transdermal delivery system and create a much more effective delivery for this water. The “polar” phase will now look like this: When you react magnesium with an acid you get a salt which is magnesium malate. As an example, to achieve about 3.0 parts per million in the H2 water: In a bottle-add 2 grams of malic acid, 4 magnesium rods, add boiling water (not distilled water) to create the hydrogen and cap bottle. Start bringing the temperature down for maximum solubility-ice bath or freezer-do not let freeze, no longer than an hour or 1.5 hours. Now use this water for the polar phase of our liposome development. Add to the area where we discuss cancer for why this hydro water is so effective. . . . Transdermal Liposomal hydro water-tie to the cancer paragraph plus drug delivery. Pure filtered water instead of distilled. Magnesium sticks, also known as Mg-rods, are one of the cheapest ways if not the cheapest way to produce H2 water. This method is a legitimate way to produce hydrogen-rich water and has been used in some research studies. These sticks consist of pure metallic or elemental magnesium (99%) and react with water to produce molecular hydrogen (Mg+2H20=>Mg(OH)2+H2)[3]. They normally require a catalyst, such as malic acid to stimulate the reaction and/or prevent passivation. If an individual uses malic acid then the reaction will also produce magnesium malate (C4H4 MgO5) as a by-product. This method is marketed to produce 1.0˜1.5 mg/L (ppm) of dissolved molecular hydrogen in 500 mL of water. The next step is the “Non-polar phase,” which is accomplished by combining, through shear mixing, any therapeutic oils with desired therapeutic oil solvents compounds. The therapeutic oils can include Coconut MCT (medium-chain triglycerides) oil, and other essential oils. The therapeutic oil solute can include but is not limited to cannaboinaoids, Vitamin D3, Vitamin B12, Vitamin E, Vitamin A, black pepper oil, zinc oxide, etc. In the next step, varying mixtures and quantities of emulsifying agents, such as gum acacia, are mixed into the therapeutic polar phase and the non-polar phase. In the next step, the “Therapeutic polar phase” is emulsified into “non-polar phase” using high pressure homogenization to form a “loaded non-polar phase.” This phase uses high pressure to force the two liquids together and break them down into small droplets, ultimately leading to the formation of an emulsion. The “Secondary polar phase” is prepared by hydrating gum acacia, and other natural agents (maltodextrin), in distilled water for a period of time, generally greater than 12 hours, to achieve desired form/thickness factor. The “Loaded non-polar phase” is then emulsified into a “secondary polar phase” using high pressure homogenization to form micelle/liposome complexes containing electrolytes and nutrients wrapped in oils.

These liposome complexes containing the electrolytes are then dried. The complexes can be dried by a variety of methods including spray drying, flake drying, oven drying. Most drying methods are appropriate as long as they form a powder that is still an oil, a proliposome. This micro encapsulation protects the therapeutic ingredients and eliminates the differences in how they interact with the skin. This allows optimal skin contact. When mixed with a water based or natural carrier, these liposome/micelle combinations become a transdermal electrolyte cream for a topical application.

In another embodiment, the invention consists of a method for preparing stable liquid emulsion forms of electrolytes, for human and animal use comprising the following steps. First, the electrolytes (or salts) are dissolved by mixing (shear mixing as one example) or heated, using the smallest particle size of each water-soluble ingredient is best since a larger surface area will create better dissolution. In this way, more electrolytes can be fully dissolved and eventually encapsulated in the liposome, and then placed into distilled water. Other desired water-soluble therapeutics that aid the body can be added in this phase as well, including but not limited to Vitamin C, Vitamin B-12, honey, Aloe vera, collagen, witch hazel, any hydrophobic drugs (for example pain medications), amino acids, and the like will be dissolved in this step as well. This step creates what will be called the “polar phase.” Next, the “Non-polar phase” will be prepared by combining through mixing and/or heating any therapeutic oil including but not limited to Coconut MCT oil, fatty alcohols, cholesterol essential oils, amino acids, cannabinoids, phospholipids for example phosphatidylcholine, and the like with desired therapeutic oil solvent compounds such as Vitamin D3, Vitamin B12, Vitamin E, Vitamin A, black pepper oil, zinc oxide, cannabinoids, or collagen. Then varying mixtures and quantities of emulsifying agents such as gum acacia, are mixed into the therapeutic polar phase and the non-polar phase. The “Therapeutic polar phase” will be emulsified into the “non-polar phase” by using high pressure homogenization to form a “loaded non-polar phase.” Next, the “Secondary polar phase” is prepared by mixing in distilled water and hydrating gum acacia, and/or other natural agents, such as maltodextrin, to achieve the desired form factor or thickness, for a period of time of greater than 12 hours. Next the “Loaded non-polar phase” is emulsified into “secondary polar phase” using high pressure homogenization to form liposome transdermal complexes containing the electrolytes wrapped in oils or lipids. During emulsification/homogenization the temperature is lowered, which can be done through the use of ice baths, chillers, cold beads, and the like, which helps prevent any heat damage to the phases.

These homogenized liposome complexes containing the electrolytes, wrapped in oils, are then dried. The liposomes can be spray dried, flake dried, or oven dried and the like, to form a powder. This powder contains the encapsulated electrolytes of this invention. This powder can be used in a wide variety of forms to introduce the electrolytes into the body in ways other than digestion. When used as creams or dissolved in water, the electrolytes are absorbed transdermally into the body. When placed in consumable or drinkable products, the liposomes are absorbed through the lining of the stomach or intestine, and are absorbed without digestion. When dissolved in water these liposome complexes can be consumed through drinking, or can be used in a bath or a soak, to introduce electrolytes. Unlike other bath soaks, for example, traditional Epsom Salts that do not fully dissolve unless heated to 130 Degrees Fahrenheit in water, and in which the particle size of most of these soaks are still too large to enter the skin, the present liposomes are manufactured to cross the dermis of the skin.

It is possible, and within the conception of the invention, to introduce the liposomes into a transdermal cream. The cream can be topically applied to specific areas of the body. Use in a cream creates the optimal skin contact, with millions of liposomes embedded in a water-based hydrator, targeting a desired area on contact. When mixed with a water based or natural carrier-these liposome electrolyte combinations become a transdermal electrolyte cream. In one embodiment the cream is created by combining distilled water (65 grams), oils (30 grams), emulsifying wax (4 grams), preservative (1 gram), and at least 9% of the disclosed liposome formula. It is also possible, and within the conception of the invention, to create a gummy for consumption, with the liposomes embedded in the pectin or gelatine based gummy. This is achieved by suspending the liposomes, and gently low shear mixing gelatin or pectin to it at low temperatures to avoid damaging the liposomes, to create an oral supplement which protects the therapeutic ingredients. The gummy eliminates the need to go through the digestive track, which helps eliminate drug-nutrient interactions. Athletes or anyone with low electrolyte levels can now avoid drinking excess amounts of electrolyte drinks, and simply have a gummy and water to achieve the desired electrolyte level. These lipids wrapping the electrolytes will immediately start to be absorbed into the body increasing the process of bioavailability. As noted above, it is possible to introduce various therapeutics into the formula, and hence the resulting liposomes, which allows this process to be used to carry these therapeutics into the body.

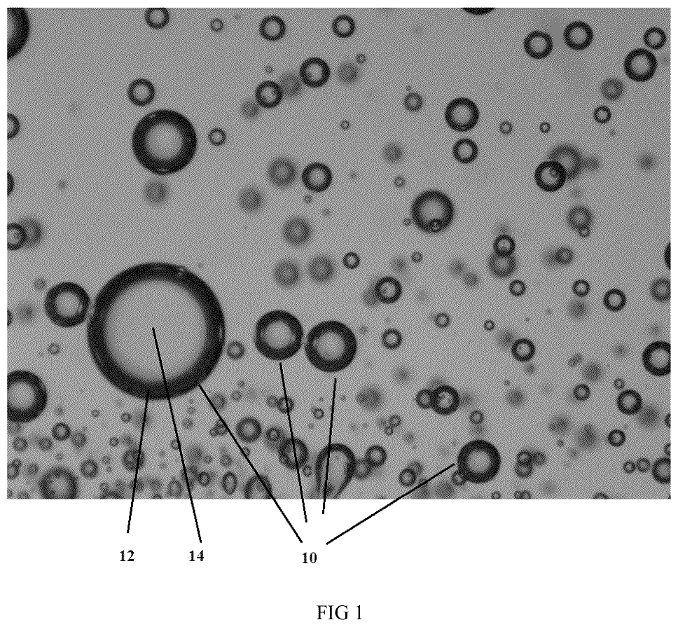

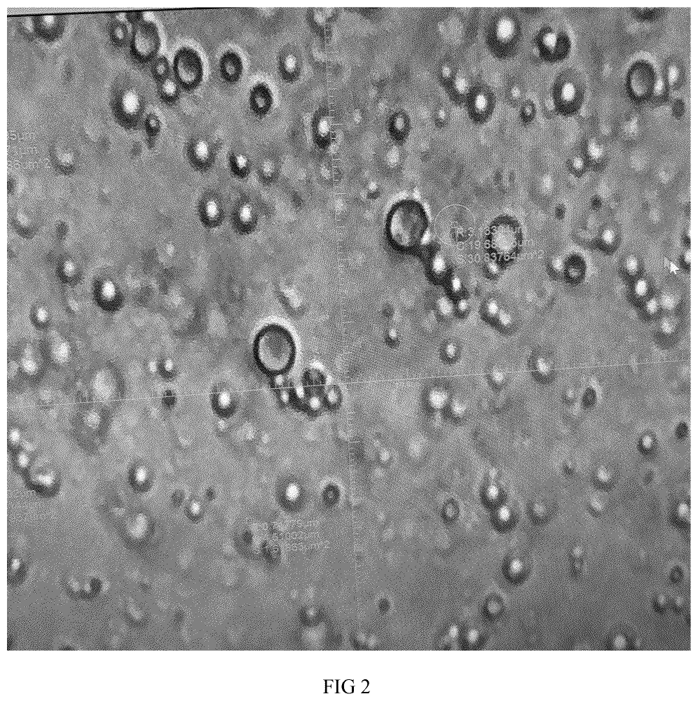

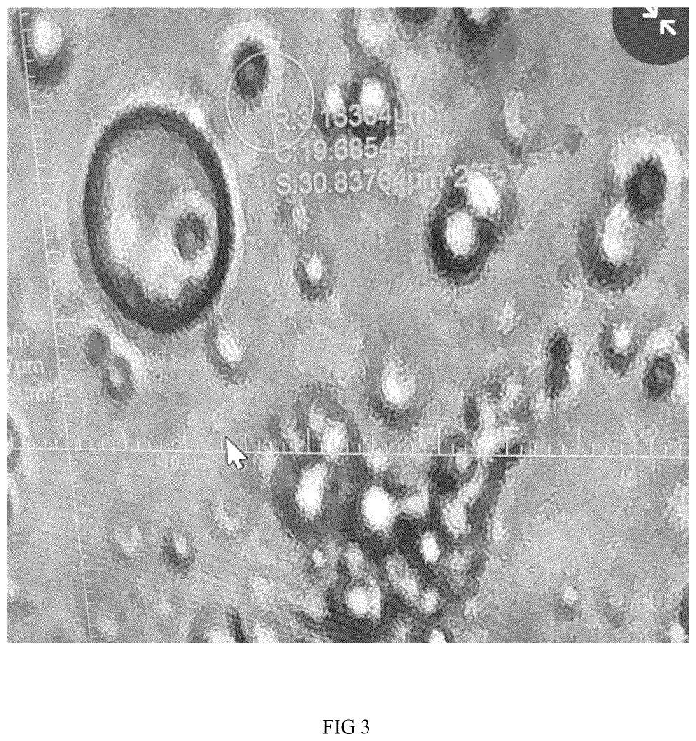

The end product liposomes are shown in FIGS. 1, 2, and 3. FIG. 1 is a detail showing the resulting liposome 10, and the components of the liposome, which are the lipid encapsulation layer 12, and the electrolyte core 14. FIG. 2 is an image from a microscope with a measurement grid imposed to show the size of the individual liposomes. As seen in FIG. 2, one of the large liposomes is shown next to a scaled circle with a radius of 3.13304 μm. It is noted that this is one of the larger particles, and most other are smaller. FIG. 3 is a larger scale microscopic image, with the same scale radius showing one of the larger liposomes 10.

1. A method of creating a liposome therapeutic electrolyte, comprising the steps of:
providing a first volume of distilled water, then dissolving maltodextrin and gum acacia in distilled water, mixing for fifteen minutes, placing the mixture in a sealable container and storing for 24 hours at a temperature below 50° Celsius to create a “water phase” solution;
providing a second volume of distilled water then dissolving Epsom salt in second volume of distilled water and mixing to create a “salty water phase” solution, then storing this at room temperature for subsequent use;
provide a warmed vessel warmed to 60° Celsius, add a volume of coconut MCT oil, then mix the desired essential oils to the warmed vessel, then mix in a volume of cannabinoids, and then begin mixing this solution, add glycerol monostearate to the solution being mixed, and continue to mix for 15 minutes to create a “oil phase” solution, keeping the solution below 80° Celsius, coving the mixture while preparing other steps;
mixing the “oil phase” solution, adding the “salty water phase” solution to the mixing “oil phase” solution, and continue mixing for at least fifteen minutes to create a “salty water in oil” emulsion, cover and store this emulsion at 60° Celsius;
homogenize the “salty water in oil emulsion in a high pressure homogenizer at between 10,000 to 20,000 psi, then add a volume of heated coconut MCT oil at 75° Celsius, then pass this mixture through the high pressure homogenizer three times;
placing the “water phase” solution in a vessel, placing the vessel into a cool bath, begin shear mixing the cooled water phase, adding the “salty water in oil” emulsion at a rate of 1 g/see, continue mixing for at least 15 minutes, remove any substance that floats to the top, to create a “salty water in oil in water” phase;
homogenize the “salty water in oil in water” emulsion in a high pressure homogenizer at between 10,000 to 20,000 psi, and passing the emulsion through the homogenizer three times, coving and storing the emulsion at 22° Celsius;
drying the emulsion to create a water soluble powdered proliposome;
wherein said proliposome can be added to a cream for a topical therapeutic or to water for a water soaking therapeutic for the transdermal delivery of the electrolyte based liposome.

2. The method of creating a liposome therapeutic electrolyte of claim 1, wherein the distilled water of the first step is provided at a temperature of between 15°-25° Celsius, and where the ingredients are provided in the ratio of 58.20% water, 17.90% gum acacia, and 13.40% maltodextrin.

3. The method of creating a liposome therapeutic electrolyte of claim 1, wherein the ingredients are provided in the ratio of 64% distilled water and 36% Epsom salt by mass.

4. The method of creating a liposome therapeutic electrolyte of claim 1, wherein the ingredients are provided in the ration of 55% coconut MCT oil, 20% CBD, 10% CBG, 5% Eucalyptus globulus leaf oil, 5% Piper nigrum Essential Oil, and 5% glycerol monostearate.

5. The method of creating a liposome therapeutic electrolyte of claim 1, wherein the “salty water phase” is added to the “oil phase” at a rate of 1 g/see, and where the mixing of the “oil phase” and the “salty water phase” occurs with a shear mixer on high.

6. The method of creating a liposome therapeutic electrolyte of claim 1, wherein the drying the emulsion can by done by spray drying, steam drying, flake drying, or dying on a sheet in an oven.

7. The method of creating a liposome therapeutic electrolyte of claim 1, replacing the distilled water of the “salty water phase,” with “hydrogen water” which is created by the following steps:
providing a vessel, introducing malic acid and magnesium rods into said vessel, then adding boiling water into said vessel, then enclosing said vessel, and cooling to above freezing, and storing for between 1 and 1.5 hours.

8. A method for preparing stable liquid emulsion forms of electrolytes for human and animal use, comprising the steps of:
dissolving electrolytes by mixing in distilled water to create a “therapeutic polar phase”;
adding other desired water-soluble therapeutics from the list of Vitamin C, Vitamin B-12, honey, Aloe vera, collagen, witch hazel, amino acids, or any hydrophobic drugs;
mixing any therapeutic oil selected from the list of Coconut MCT oil, fatty alcohols, cholesterol essential oils, amino acids, cannabinoids, and phospholipids for example phosphatidylcholine, with a desired therapeutic oil solvent compound selected from the list of Vitamin D3, Vitamin B12, Vitamin E, Vitamin A, black pepper oil, zinc oxide, cannabinoids, or collagen, to create a “non-polar phase”;
adding an emulsifying agent, such as gum acacia, to the “therapeutic polar phase” and the “non-polar phase”;
emulsifying the “therapeutic polar phase” into the “non-polar phase” using high pressure homogenization to create a “loaded non-polar phase”;
hydrating gum acacia and other natural agents such as maltodextrin in distilled water for a period of greater than 12 hours to create a “secondary polar phase”;
emulsifying the “loaded non-polar phase” into the “secondary polar phase” using high pressure homogenization, and lowering the temperature to above freezing to create liposome transdermal complexes containing electrolytes wrapped in oils;
drying the liposome transdermal complexes by spray drying, flake drying, oven drying, to form a powderized proliposome that can be further used in other forms.

9. The method for preparing stable liquid emulsion forms of electrolytes for human and animal use of claim 8, comprising the further steps of:
combining the powderized proliposome with distilled water, oils, and emulsifying wax to create a liposome cream.

10. The method for preparing stable liquid emulsion forms of electrolytes for human and animal use of claim 8, comprising the further step of:
combining the powderized proliposome with gelatin or pectin to create liposome rich edible gummy for human consumption.

11. The method of creating a liposome therapeutic electrolyte of claim 1, wherein the steps of mixing is done by blending with an emulsifier at high pressure.

12. The method of creating a liposome therapeutic electrolyte of claim 3, wherein the Epsom salt can be replaced by any appropriate electrolyte chosen from the list of dead sea salt, magnesium chloride or potassium.
